(19) United States
(12) Patent Application Publication
(10) Pub. No.: US 2022/0125833 A1
(43) Pub. Date: Apr. 28, 2022

(54) VITAMIN D COMPOSITIONS AND METHODS OF USE IN THE TREATMENT OF COVID-19 AND OTHER LIPID ENVELOPED VIRAL INFECTIONS

(71) Applicant: Kenneth Pickens, Omaha, NE (US)

(72) Inventor: Kenneth Pickens, Omaha, NE (US)

(21) Appl. No.: 17/451,843

(22) Filed: Oct. 22, 2021

Related U.S. Application Data
(60) Provisional application No. 63/104,669, filed on Oct. 23, 2020.

(51) Int. Cl. A61K 33/30; A61K 31/59; A61K 31/661; A61K 31/4045; A61K 31/355; A61P 31/14

(52) U.S. Cl. CPC A61K 33/30; A61K 31/59; A61P 31/14; A61K 31/4045; A61K 31/355; A61K 31/661

(57) ABSTRACT

Vitamin D formulations and method of administering the same for COVID-19 patients.

### CROSS REFERENCE TO RELATED APPLICATIONS

This application claims priority to U.S. Provisional Application No. 63/104,669 filed Oct. 23, 2020, the entire contents of which are incorporated herein by reference.

### FIELD OF THE INVENTION

The present invention relates to Vitamin D, and Vitamin D and phospholipid, formulations for the disruption of SARS-CoV-2 and other lipid envelope containing viruses, and more particularly, Vitamin D, and Vitamin D and phospholipid, formulations and methods of use in the treatment and prevention of COVID-19 in patients in need thereof.

### BACKGROUND OF THE INVENTION

The SARS-CoV-2 virus has resulted in a worldwide pandemic, being lethal in about 1-3% of infected individuals. Although there are several approved vaccines, individuals are still becoming infected, and the virus still remains a significant health threat. The predominant mediation measure for the virus in most areas of the world is social distancing, face coverings, and hand washing meant to facilitate a slowing of the spread of the virus. For individuals that become infected with SARS-CoV-2, the virus tends to result in a myriad of symptoms, from mild respiratory and common cold problems to pneumonia and respiratory failure. Other symptoms can include gastrointestinal problems, blood clots, fever, headache, loss of taste and smell, and septic shock, to name just a few. There is a need in the healthcare industry for treatments that can prevent or mediate the severity of a SARS-CoV-2 infection.

Against this backdrop the present disclosure is provided.

### SUMMARY OF THE INVENTION

Embodiments of the present invention include formulations having fat soluble vitamins, and in particular, formulations that include Vitamin D, where the formulations can also include various sized nanoparticles. In some aspects, the formulations can further include various phospholipids.

In some cases, the formulations described herein are used to inhibit SARS-CoV-2 viral replication (or other lipid envelope containing viruses), and in some cases, the formulations can be used to prevent or treat COVID-19 in individuals in need thereof. Individuals can be infants, youth, young adults, adults, or the elderly. Formulations can be administered by various conventional routes, and include oral tablets, capsules, as a food, IV or injection. Formulations can also include nanoparticles, zinc, melatonin and/or phospholipids. Where the single dose of Vitamin D is above 25,000 IU, administration is typically via IV or injection. In some instances, very high doses of Vitamin D can be used for short periods of time, including up to 300,000 IU.

### DETAILED DESCRIPTION OF THE EMBODIMENT OF THE INVENTION

Enveloped viruses like SARS-CoV-2 tend to be susceptible to deactivation because they are protected with a lipid envelope. These lipid coated viruses, with this lipid wrapping, are relatively vulnerable to soap, for example, when you wash your hands. Disruption of the lipid envelope results in the virus losing its capacity to invade and replicate in target cells.

The dissolution of a nonpolar solute such as lipid envelope can be accomplished spontaneously through the use of a nonpolar solvent. The four vitamins that are fat-soluble (soluble in nonpolar solvents) are Vitamins A, D, E, and K. Embodiments herein include the use of Vitamin A, D, E and/or K for the prevention or treatment of COVID-19. It is the inventor's hypothesis that the presence of these vitamins, in sufficient quantities, in a subject's blood and cellular compartments, will lower the viral load by interfering with the virus's lipid envelope (the Vitamin D and other fat soluble vitamins are incorporated into cells where they continue to have their oil state). Disruption of the viral envelope in a subject will thus lessen the likelihood of that subject contracting COVID-19, or at least lowering the viral load, creating a less serious case of COVID-19 when receiving these supplements.

Vitamin D, in particular, shows excellent properties and capacity for dissolving the virus envelope because it is oil solvable, and the vitamin is held together by a weak van der Waals interaction. Vitamin D is a hormone having an active form of 1α,25-dihyroxyvitamin D (1α,25(OH)2D). The vitamin is considered a secosteroid with a broken 9, 10 carbon-carbon bond in the B ring of the cyclopentanoperhydrophenanthrene structure, which is a carbon skeleton found in steroids.

Although only circumstantial evidence regarding the use of Vitamin D for COVID-19 treatment, for many Americans the main source of Vitamin D is found in milk. But millennials are known to be among the lowest consumers of dairy products, including milk. Conversely, millennials are at a higher risk for coronavirus infection, compared to any other age population, except people who are immunocompromised, or those having an age over 70 years old. Through early statistics, women appear to be half as likely to contract the virus as men. Further, women's blood is less acidic than men, as are smokers, they are in general more acidic. This makes it easier to break down water solvable bonds in the blood, but less able to break down oil solvable bonds. Obvious pH levels cannot vary that much between males and females but it may be enough to create the thermodynamic energy needed to dissolve or tamper with the lipid core of the virus, explaining why men are twice as prone to be infected with the virus as are women.

In the blood stream, the SARS-CoV-2 virus, after replicating in a host cell, circulates in search of other host cells to invade. The virus is held together or enveloped by a weak lipid or fatty membrane. Due to the reason set forth hereafter a change in the enthalpy charge in the blood will spontaneously dissolve the protective coating of the virus. It is through the use of fat soluble vitamins that this enthalpy charge can be altered in the blood.

The nonpolar solute fat molecules that encapsulate the coronavirus core and preserve its structure are held together by weak van der Waals interactions due to the small size of the virus and the chemical composition of the virus. While the van der Waals interaction gives it a semi-stable lipid shell, it is reported, consistent with van der Waal reactions, that the virus spontaneously dissolves when a person washes their hands in soap and water. The enthalpy change, (a thermodynamic quantity equivalent to the total heat content of a system is equal to the internal energy of the system plus the product of pressure and volume), required to break the viral interactions is therefore small. By adding Vitamin D, or the other fat soluble vitamins, which is held together by a van der Waals interaction, it would act as a nonpolar solvent molecule. Despite vitamin D being held together only by weak van der Waals interactions, the enthalpy change would also be small. Even though the solute and solvent particles will also not form strong interactions with each other there is very little energy required to break these virus particles.

Hence, the overall enthalpy change (DH1+DH2+DH3) is small. The small enthalpy change (DH), together with the positive entropy change (a thermodynamic quantity representing the unavailability of a system's thermal energy for conversion into mechanical work) for the process (DS), result in a negative free energy change (DG=DH−TDS) for the process; hence, the dissolution of the virus in the presence of the fat soluble vitamins occurs spontaneously. It is also noted that vitamin D, in particular, holds promise in other ways as it supports lung function. In particular, Vitamin D (and Vitamin A as well) acts in a barrier function manner in mucous membranes in the respiratory tract. For example, Vitamins D acts to inhibit the production of pro-inflammatory cytokines and increases the production of anti-inflammatory cytokines.

In one embodiment, the fat soluble vitamins are formulated for oral intake and ingested as tablets, capsules, as a food or liquids; in another embodiment, the vitamins are formulated for administration by IV or injection, thereby avoiding the acidic environment of the stomach. It is believed that IV administration or injection of the vitamins will maximize the vitamin's van der Waals interactions with the virus (discussed more below).

Embodiments herein include ingestion of larger oral doses of the oil based vitamins (typically above 25,000 IU), which would be carried by the blood stream. Where large doses are used, the vitamins are typically administered by daily, weekly or monthly injection or IV infusion. In some instances, up to 300,000 IU can be administered of these oil based vitamins.

In alternative embodiments, Vitamin D (or the other fat soluble vitamins) is combined with lecithin and a polymer and then micro fluidized into nanoparticles. This formulation allows the Vitamin D to become time released, and the mass of the nanoparticle would by nature make the likelihood of the viral weak van der Waals attraction being drawn to the vitamin D weak van der Waals attraction, causing the virus to dissolve. In some embodiments, the nanoparticles are from approximately 1 to 2,000 nm in diameter, and more typically 15 to 500 nm, and still more typically 15 nm to 250 nm in diameter. In other embodiments, the nanoparticles are from approximately 15 to 75 nm in diameter. Embodiments include: Vitamin D, one or more polymers, lecithin and nanoparticles, Vitamin E, one or more polymers, lecithin and nanoparticles, Vitamin A, one or more polymers, lecithin and nanoparticles, and Vitamin K, one or more polymers, lecithin and nanoparticles. The one or more polymers are of a pharmaceutical type and grade. Embodiments also include combinations of Vitamins D, E, A and/or K, one or more polymers, lecithin and nanoparticles.

In addition, embodiments herein can include any of the fat soluble vitamins combined with melatonin. This combination of active ingredients can be combined with lecithin and a polymer. In some cases, the fat soluble vitamin, melatonin, lecithin and polymer an be micro fluidized into nanoparticles of approximately 15 nm to 250 nm in diameter.

In addition, embodiments herein can also include any of the fat soluble vitamins with magnesium to enhance absorption of the vitamins. In one particularly useful embodiment, the formulation includes Vitamin D, Vitamin K, magnesium and zinc. In another particularly useful embodiment, the formulation includes Vitamin D, Vitamin K, magnesium, zinc and melatonin.

These vitamin containing compositions do not require FDA approval as the ingredients are all individually FDA approved. In one proposed experiment, the SARS-CoV-2 virus could be placed into a Petri dish with human blood, taken from a person that had been administer Vitamin D (or Vitamin D3) over a sufficient time so that their blood samples showed they had elevated, but safe, Vitamin D3 levels. You could then observe the effects of the elevated Vitamin D3, while keeping the blood at body temperature, how long for the viral lipid envelops to begin to dissolve.

You would do a control study using a person's blood with normal levels of Vitamin D3 and virus under the same test conditions to determine how long it would take those lipid envelops to begin to dissolve.

The key to the inventor's findings disclosed herein is that due to van der Waals interactions, Vitamin D (or the other fat soluble vitamins) will spontaneously dissolve the SARS-CoV-2 lipid envelop around the virus when it comes into its proximity, with dissolution of the envelope destroying the virus. Melatonin facilitates and synergistically enhances the dissolution of the SARS-CoV-2 lipid envelop around the virus.

Embodiments herein rely on the rule in chemistry that says “that ‘like dissolves like’ (Allen Blackman, PhD, for example). In this case, Vitamin D and other fat soluble vitamins dissolve the lipid envelope of SARS-CoV-2.

The dissolution of a nonpolar solute such as the lipid envelope on SARS-CoV-2 is done spontaneously by Vitamin D3, as it is a nonpolar solvent. Taken orally, or by injection or IV, Vitamin D would have the potential for those not yet infected in assisting their immune system once they are exposed to the virus by rapidly eliminating any viral load and in assisting the immune system for those that are ill by rapidly reducing viral load.

Enveloped viruses like SARS-CoV-2 rely on a protective lipid (oil based) envelop and are the easiest type to collapse because they are held together by these weak lipid envelopes. The coronavirus has a nonpolar solute lipid envelop that is vulnerable to a nonpolar solvent.

### Findings Based on Theory of Van Der Waal Interactions

As noted above, the nonpolar solute fat molecules that encapsulate the coronavirus virus and preserve its structure are held together by weak van der Waals interactions which create an unstable lipid shell around the virus. The enthalpy change (a thermodynamic quantity equivalent to the total heat content of a system is equal to the internal energy of the system plus the product of pressure and volume) required to cause the collapse of this envelop structure is very small. This has been noted by the use of plain soap and water to sanitize an exposed persons hands, due to the fact that soap and water act as a surfactant or amphiphilic compound which disrupts the viral envelope.

Vitamin D is held together by van der Waals interactions. By adding Vitamin D to a subject's system, the Vitamin D is attracted to the SARS-CoV-2 virus (both are non-polar). The degree of attraction is based on the distance between the virus and the interacting atoms of Vitamin D. Upon coming into close proximity, the Vitamin D or other fat soluble vitamin, would act as nonpolar solvent molecule.

Despite Vitamin D being held together by weak van der Waals interactions, there would be a small enthalpy change when the Vitamin D came into contact with the lipid envelop of the virus. Even though the solute and solvent particles do not form strong interactions with each other, there is very little energy required for a reaction to occur.

When the Vitamin D van der Waal attraction is attracted to the SARS-CoV-2 virus van der Waal attraction, and is in proximity with the lipid on the virus, an overall enthalpy change starts which would be small but enough to start the dissolution process. The small enthalpy change is created by the vitamin D (or other fat soluble vitamin) and results in a positive entropy change for the lipid structure, which then causes a negative free energy that results in a dissolution of the lipid envelop surrounding the virus to occur spontaneously.

### Issue of Energy Stability

The smaller the particle size, with a mass that correlates to a nanoparticle, the closer in proximity it can come to the lipid envelop of the virus, and the closer the proximity, the greater the probability of spontaneous dissolution of the lipid envelop. The in vitro studies showed that smaller lipid droplets are digested more rapidly than larger ones, and the highest Vitamin D bioavailability was observed for the emulsions containing the smallest droplets. This would certainly be beneficial for most Americans who are Vitamin D deficient and need Vitamin D for its known properties of being an immune-stimulator and enhancer of lung function. Vitamin D is also known to have receptors in bone, gut and kidney, which each could benefit from Vitamin D ingestion.

The issue becomes, is the reduction in size of the Vitamin D particles decrease the energy stored for the transfer of energy to the lipid envelop sufficient to cause a negative effect on the simultaneous dissolution of the virus or is it a positive or neutral event. It would depend in part by the process used, and the size and mass of the particle relative to the size of the and mass of the lipid envelop around the virus.

Food manufactures have reported the use of nanoemulsion mixtures of Vitamin D in food products to fortify them in colloidal dispersions comprising two immiscible liquids with droplet sizes, and corresponding mass, of the internal phase between 1 and 200 nm, and more typically 50 and 175 nm

The SARS-CoV-2 virus by size comparison has a round or elliptic shape and often pleomorphic form, and a diameter of approximately 60-140 nm. The diameter however is not made up entirely of a solid lipid mass like the oil found in the Vitamin D droplet but instead the lipid envelop is described as a thin outer layer and therefore lacks the mass of the Vitamin D containing nanoparticle.

As such, some embodiments herein include administration of up to 25,000 IU Vitamin D per day, up to 25,000 IU Vitamin K, up to 25,000 IU Vitamin E or up to 25,000 Vitamin A to an adult subject. In some aspects, an adult subject is administered, on a daily basis, 1,000 IU, 2,000 IU, 3000 IU, 4,000 IU, 5,000 IU, 6,000 IU, 7,000 IU, 8,000 IU, 9,000 IU, 10,000 IU, 11,000 IU, 12,000 IU, 13,000 IU, 14,000 IU, 15,000 IU, 16,000 IU, 17,000 IU, 18,000 IU, 19,000 IU, 20,000 IU, 21,000 IU, 22,000 IU, 23,000 IU, 24,000 IU or 25,000 IU of any one of, or a combination of, Vitamin D, Vitamin K, Vitamin E and Vitamin A In other embodiments, the administration of one or more of Vitamins A, D, K or E can be up to 50,000 IU/day. These higher doses can be a combination of one or more of the Vitamins, for example, 30,000 IU Vitamin D and 20,000 IU Vitamin A, or all of a single Vitamin, for example, 50,000 IU Vitamin D. In still other embodiments, very high amounts of a fat soluble vitamin can administered, in one aspect, up to 100,000 IU, and in some cases, up to 200,000 IU, and in still other cases, up to 300,000 IU. In these higher amounts, a shorter course of administration may be proper.

The administration can be orally or via IV or injection. In some cases, the Vitamin D, Vitamin K, Vitamin E or Vitamin A is combined with colloidal carries (range of 10⁻⁹m) to enhance the vitamins bioaccumulation and bioavailability. Colloidal carries herein include liposomes, polymeric NPs, or dendrimers having a mass corresponding to the mass of at least the viral envelope. In some embodiments the carrier is a nanoparticle composed of poly(D,L-lactide-co-glycolide (PLCA) or liposomes composed of multilamellar vesicles (MLVs). Nanoparticles allows for the higher amounts of fat soluble vitamins to be administered, but it also allows for increased bioavailability and a minimalization of toxicity.

In another embodiment, the vitamins (D, K, E and A) could also be combined with lecithin or polymer combinations to form a microemulsion and be administered via microfluidazation, again with a target mass of at least the mass of a viral particle.

In other embodiments, one or more of the Vitamin D, Vitamin K, Vitamin E or Vitamin A are combined on a daily basis to provide the 1,000 IU to 50,000 IU to the adult subject. For example, a subject could ingest 5,000 IU of Vitamin D and 5,000 IU of Vitamin E when combined with a colloidal carrier, for a total dose of 10,000 IU fat soluble vitamin. Embodiments also include administration of a fat soluble vitamin to an infant of one year or less, one year to three years, four years to eight years, and nine years and above. For infants, embodiments include administration of from about 400 IU to about 1,000 IU Vitamin D or other fat soluble vitamin. For children between the ages of one and three, administration of from about 400 IU to about 2,500 IU Vitamin D or other fat soluble vitamin. For children between the ages of four and eight, from about 400 IU to about 3,000 IU and for children aged 9 to young adult, from about 400 IU to about 6,400 IU of Vitamin D or other fat soluble vitamin.

For each of the above embodiments, zinc, zinc acetate or zinc gluconate can be combined with the fat soluble vitamins and/or colloidal particles to have a vitamin, nanoparticle and zinc formulation. Zinc compounds can be added at from about 5 mg to 15 mg per daily dose, but can be dosed according to the subject's gender, 8 mg/day for women and 11 mg/day for men.

For each of the above embodiments, melatonin may also be combined with the fat soluble vitamins, zinc (zine acetate or zinc gluconate) and/or colloidal particles to have a formulation. Melatonin can be added at from about 0.5 mg to about 10 mg per daily dose, but can be dosed according to the subject's gender and weight.

### Phospholipids and Vitamin D

To increase the bioavailability of vitamin D3 to get in close proximity to the virus in a person, phospholipids have excellent biocompatibility for inclusion in formulations herein. In addition, phospholipids are renowned for their amphiphilic structures. Phospholipids have a propensity to form liposomes which can be employed as a drug carrier for the vitamin D or other fat soluble vitamins.

Various phospholipids, such as soybean phosphatidylcholine, egg phosphatidylcholine, or synthetic phosphatidylcholine, as well as hydrogenated phosphatidylcholine, can be used in these formulations.

Nanotechnology platforms using phospholipids in the area of medical biology therapy, have gained attention. Moreover, research and development of engineered multifunctional nanoparticles as pharmaceutical drug carriers have spurred exponential growth in applications to medicine in the last decade, and would be one method to deliver Vitamin D3 to close proximity to the SARS-CoV-2 lipid envelop. In the process, it could either initiate a van der Waals spontaneous dissolution of the virus' lipid envelop or because the water in blood is a polar molecule, while lipid envelop is a nonpolar molecule, there is some separation of charge. One end of the molecule is slightly positively charged and the other is negatively charged.

The polar molecules in the blood can have both a positive and a negative end, just as a battery does, while a nonpolar molecule has its charge evenly spread.

There's a rule in chemistry that ‘like dissolves like.’ Polar molecules ‘like’ other polar molecules . . . so polar molecules will dissolve in water.” Try and wash anything oily or greasy off, the nonpolar lipid envelop is insoluble in blood which is primarily water.

Add Vitamin D which is fat soluble and delivered by the phospholipids, however, and the lipid envelop of the virus will dissolve away.

That's down to the unique molecular structure of phospholipids, and it works just as well against a virus protected by a fatty envelope as it does against any greasy object. Combinations of the phospholipids with the Vitamin D (and nanoparticles) maximizes the capabilities of these materials to disrupt the virus particles in the blood.

Liposomes as carriers of therapeutic drugs have attracted attention for more than 30 to 40 years. Liposomes have many advantages for delivering both hydrophilic and lipophilic drugs, possessing targeting, controlled release properties, cell affinity, tissue compatibility, reducing drug toxicity and improving drug stability. The conventional structures of the liposomes have some changes, which have brought out a series of new type liposomes, such as long-circulating liposomes, stimuli-responsive liposomes, cationic liposomes and ligand-targeted liposomes. In this light, formulations that include fat soluble vitamins, liposomes and zinc are contemplated.

In some of these aspects, the Vitamin D added to the phospholipid would create a lipid bilayer. Non-bilayer lipids such as unsaturated PE can be stabilized in a bilayer structure by the presence of bilayer preferring lipids such as PS. It is usually found that between 20 and 50 mol % of the bilayer preferring lipids are required to maintain a net bilayer organization when mixed with HII preferring lipids such as PE. The structural preferences of these pure and mixed lipid systems can be modulated by a wide variety of factors, such as head group size, temperature, hydrocarbon unsaturation, ionic strength, pH, the incorporation of inverted cone molecules, and the presence of divalent cations such as Ca²⁺.

The smaller head group of PE (as compared to PC) is consistent with HII organization. In addition, in the case of PE, increasing acyl chain unsaturation, temperature and acyl chain length lead to increased cone shape and possible HII phase formation. The lamellar to HII transition temperature (Tbh) decreases with the increasing chain length and the increasing unsaturation of acyl chain of PE. Many species of naturally occurring PE preferentially adopt a HII phase at physiological temperatures.

The phospholipid head size would make the delivery of vitamin D to the SARS-COV-2 more likely, before its degradation by the body. In other aspects, intravenous emulsions originated from the development of intravenous nutrition emulsions. In 1962, Intralipid was successfully developed in Sweden and proved to be safe and effective, which lay the foundation for the development of medicinal intravenous emulsions. Intravenous fat emulsions possess many advantages, including targeting, reducing drug toxicity, which make them attract more and more attentions recently.

Phospholipids as zwitterionic surfactants can be used as emulsifying agent of oil-in-water (O/W) type emulsions, and due to their biological and non-toxic characteristics, they can be used as emulsifiers for intravenous injection. The O/W type emulsions are mainly composed of two parts: the oil core and the emulsifying agents on the surface. Unlike liposomes, fat emulsions are suitable for large-scale industrial production and relatively stable below 25° C. for long term More importantly, a large quantity of lipophilic drugs can be dissolved in the hydrophobic core of emulsions. In such cases, the fat soluble vitamin is dissolved in the hydrophobic core of emulsions, and can be formulated for various doses of the vitamin.

Methods herein include use of vitamin formulations to treat a SARS-CoV-2 infection in a subject in need thereof. Methods include identifying a subject (typically a human) that is in need of one of the formulations described herein. Identification can include testing the subject for the presence of the SARS-CoV-2 virus; where positive, determining the overall health status of the subject and whether the subject requires a high or mega dose of the fat soluble vitamin (above 20,000 IU to 25,000 IU, and up to 300,000 IU); determining what type of formulation, in accordance with embodiments herein, the subject should receive; and receiving the formulation through an appropriate route (where the Vitamin is a mega dose, the administration would be IV or injection). In some embodiments, the administration can be once per day, once every two days, once every three days, once every four days, once every five days, once every six day, or once a week. In some aspects, the methods include administering the vitamin formulations to subjects that are infants (younger than one), subjects aged 1 to 3, subjects aged 4 to 8 and children aged 9 to 16.

In other methods, the use of the formulations can be for prevention of a SARS-CoV-2 infection. A subject in need of enhance protection from SARS-CoV-2 (health care workers, the elderly, those who have underlying health issues like heart disease, diabetes, lung disorders, etc . . . ) receives a formulation in accordance with embodiments herein, through an appropriate administration route. The subject would then receive the formulation once per day, once every two days, once every three days, once every four days, once every five days, once every six day, or once a week. In some embodiments, the subject would receive a test on a planned schedule to test for the presence of SARS-CoV-2.

In still other embodiments, a method is provided to treat body fluids with a fat soluble vitamin formulation. The body fluid/material could be a subject's blood, cells, an organ for transplant, in vitro tissue culture cells, and other like ex vivo treatment. The body fluid/material would be contacted with a formulation in accordance to embodiments herein.

In some aspects, the administration route for a 1-20,000 IU dose of vitamin formulation can be through oral ingestion, IV, injection or other like target. Where the administration is over 20,000 IU of fat soluble vitamin, the dose is best administered by IV or via injection. In some embodiments, the dose of vitamin is combined with a mass of appropriately sized nanoparticles and, where necessary, a phospholipid. In some embodiments, the fat soluble vitamins are combined with phospholipid and further incorporated into a food for ingestion by the subject. In some cases the fat soluble vitamin is Vitamin D and the phospholipid is lecithin. In other cases, the phospholipid is phosphatidylcholine. The phospholipid can be present in from about 0.1 g to about 5 grams, and more typically 0.4 g to about 4 g. Additionally, as noted above, compounds like zinc and Melatonin can be combined with the fat soluble vitamin, in the amounts previously stated above.

### Other Lipid Enveloped Viruses

Embodiments herein are also effective in the treatment or prevention of viruses beyond SARS-CoV-2. For example, any virus having a lipid envelope can be treated using the fat soluble vitamin formulations discussed herein, including the use of appropriately sized nanoparticles. For example, influenza virus infection can be treated using administration of Vitamin D, zinc and melatonin, for example. The principles discussed above, apply to any virus that utilizes a lipid envelope, for example, the embodiments herein can be used to prevent or inhibit replication of poxviruses, retroviruses, rhabdoviruses, flaviviruses, and herpesviruses, for example. As such, the formulation and methods discussed herein can also be used to prevent or inhibit infection of any lipid enveloped virus in a subject. Doses and combinations above, including nanoparticles, would be the same as discussed above for SARS-CoV-2.

Those skilled in the art will recognize or be able to ascertain using no more than routine experimentation, many equivalents of the specific embodiments of the invention described herein. Such equivalents are intended to be encompassed by the following claims.

Entire contents of all non-patent documents, patent applications and patents cited throughout this application are incorporated by reference herein in their entirety.

1. A dose kit comprising a dose of Vitamin D and a dose of zinc, wherein the Vitamin D and zinc are formulated with lecithin and a polymer and the dose of Vitamin D and zinc are for treating or prevention of SARS-CoV-2 virus infection.

2. A dose kit comprising a dose of Vitamin D and a dose of zinc, wherein the Vitamin D and zinc are formulated with a colloidal carrier and the dose of Vitamin D and zinc are for treating or prevention of SARS-CoV-2 infection.

3. The dose kit of claims 1, wherein the dose of Vitamin D is at least 10,000 IU and the dose of zinc is from about 5 mg to about 15 mg.

4. The dose kit of claim 3, wherein the dose of Vitamin D is at least 50,000 IU and the dose of zinc is from about 5 mg to about 15 mg.

5. The dose kit of claims 3, further comprising one or more phospholipids.

6. The dose kit of claim 5, wherein the phospholipid is lecithin.

7. The dose kit of claim 6, further comprising a dose of Vitamin K, Vitamin E, and/or Vitamin A.

8. The dose kit of claim 1, further comprising a dose of Melatonin.

9. The dose kit of claim 8, wherein the dose of Melatonin is from about 0.5 mg to about 10 mg.

10. A method of treating a subject having COVID-19 comprising:
confirming that the subject has COVID-19;
administering to the subject a formulation of at least 25,000 IU Vitamin D;
wherein the subject is administered the formulation each day until the subject no longer tests positive for COVID-19.

11. The method of claim 10, wherein the formulation further comprises zinc and at least one phospholipid.

12. The method of claim 11, wherein the formulation further comprises lecithin.

13. The method of claim 10, wherein the formulation further comprises Melatonin at a dose of from 0.5 mg to 10 mg.

14. The method of claim 10, wherein the administering the formulation is of at least 50,000 IU of Vitamin D.

15. The method of claim 10, wherein the administering the formulation is of at least 300,000 IU of Vitamin D.

16. The method of claim 10, further comprising administering an additional fat soluble vitamin.

17. The method of claim 16, wherein the fat soluble vitamin is Vitamin E.
